(19) United States
(12) Patent Application Publication
(10) Pub. No.: US 2001/0003802 A1
(43) Pub. Date: Jun. 14, 2001

(54) Magnetic spring and ventricle assist device employing same

(76) Inventor: Nicholas G. Vitale, Albany, NY (US)

Correspondence Address: HESLIN & ROTHENBERG, PC, 5 COLUMBIA CIRCLE, ALBANY, NY, 12203

(21) Appl. No.: 09/761,436

(22) Filed: Jan. 16, 2001

Related U.S. Application Data
(63) Continuation-in-part of application No. 09/382,143, filed on Aug. 24, 1999, now Pat. No. 6,190,409.
Application No. 09/382,143 is a division of application No. 08/885,142, filed on Jul. 1, 1997, now Pat. No. 5,984,960.
Application No. 08/885,142 is a division of application No. 08/640,172, filed on Apr. 30, 1996, now abandoned.

(51) Int. Cl. A61M 1/12

(52) U.S. Cl. 623/3.11; 623/3.19; 600/16

(57) ABSTRACT

A magnetic spring includes a plurality of spaced-apart stationary circumferentially magnetized segments disposed along a circle about an axis to define a first plurality of spaced-apart gaps, and a plurality of spaced-apart moveable circumferentially magnetized segments disposed along the circle to define a second plurality of spaced-apart gaps. Each of the plurality of moveable magnetized segments is axially slidable within a respective one of the first plurality of gaps defined by the plurality of stationary magnetized segments. Significant applications of the magnetic spring in an actuator of a ventricle assist device (VAD) or a total artificial heart (TAH) in which stored energy in the magnetic spring is used to reduce motor power loses of an actuator during a power stroke of the VAD or TAH.

### RELATED APPLICATION

This application is a continuation-in-part patent application of related commonly assigned, co-pending U.S. patent application Ser. No. 09/382,143, filed Aug. 24, 1999, entitled “Rotary Torque-To-Axial Force Energy Conversion Apparatus,” which has issued as U.S. Pat. No. ______ and which is a divisional patent application from prior U.S. patent application Ser. No. 08/885,142 which has issued as U.S. Pat. No. 5,984,960 which itself is a divisional of U.S. patent application Ser. No. 08/640,172 which application is now abandoned. The entire contents of each of these applications are incorporated herein by reference.

### FIELD OF THE INVENTION

This invention relates generally to springs, and more specifically, to magnetic springs. Significant applications of the magnet springs is in ventricle assist devices and in total artificial hearts.

### BACKGROUND OF THE INVENTION

Ventricle assist devices (VAD) and total artificial hearts (TAH) conventionally employ an actuator for forcing blood from the single chamber in the VAD or from the two chambers in the TAH. Typically, various rotary to linear conversion mechanisms such as a lead screw, or a gear pump and a hydraulic piston pump, are used to move a pusher plate to squeeze blood from the VAD or TAH.

For example, the Cleveland Clinic—type TAH conventionally employs an electrohydraulic energy conversion apparatus. This apparatus comprises a brushless DC motor which turns a gear pump that provides hydraulic flow at about 100 psi. Internal valving controls flow to a double-ended hydraulic actuator. To ensure that the system is hermetically sealed, the actuator piston is actually a stack of magnets riding in the cylinder, with a follower magnet outside the cylinder to match piston motion. The follower magnets are attached to a translating element that presses against a pusher plate that deflects a rubber diaphragm.

The hydroelectric actuator includes a load-biasing coil spring located in the interventricular space of the TAH. During ejection from the left side, the spring assists the follower assembly to work against systemic arterial afterloads. Also, work is required to compress the spring during ejection from the right side. This actuator is further described in Massiello et al., “The Cleveland Clinic—Nimbus Total Artificial Heart,” Journal of Thoracic and Cardiovascular Surgery, Vol. 108, No. 3, pp. 412-419 (1994) and in Harasaki et al., “Progress in Cleveland Clinic—Nimbus Total Artificial Heart Development,” ASAIO Journal, M494-M498 (1994).

Limitations in the use of a coil spring in the actuator include the following:

1) The spring produces its greatest boost at the start of eject, sometimes overrunning the actuator and producing high acceleration forces;

2) The spring assist is least at the end of the eject cycle where it was most needed; and

3) The spring can fret against the actuator shell if the spring was incorrectly assembled or buckled sideways during operation.

Magnetic springs have been used for supporting a force and typically include two concentric magnetized rings or two identically sized magnetic rings which face each other about an axis. The magnetic field or flux is primarily in a radial direction (e.g., concentric rings) or primarily in an axial direction (e.g., rings facing each other). Magnetic bearings have also been used for axially supporting a shaft and opposing axial and radial movement of the shaft. For example, a magentic spring typically includes a first set of magnets are attached to the shaft and a second set of magnets are fixedly supported at an outer distance from the first set of magnets.

There is a need for improvements in magnetic springs and in improvements in ventricle assist devices and total artificial hearts.

### SUMMARY OF THE INVENTION

The above-mentioned needs are met by the present invention which provides, in a first aspect, a magnetic spring which includes a plurality of spaced-apart stationary magnetized segments defining a first plurality of spaced-apart gaps, a plurality of spaced-apart moveable magnetized segments defining a second plurality of spaced-apart gaps, and wherein each of the plurality of moveable magnetized segments is slidable within a respective one of the first plurality of gaps defined by the plurality of stationary magnetized segments.

In a second aspect, a magnetic spring includes a plurality of spaced-apart stationary magnetized segments disposed along an arc about an axis and defining a first plurality of spaced-apart gaps, a plurality of spaced-apart moveable magnetized segments disposed along the arc and defining a second plurality of spaced-apart gaps, and wherein each of the plurality of moveable magnetized segments is axially slidable within a respective one of the first plurality of gaps defined by the plurality of stationary magnetized segments and wherein each of the plurality of stationary magnetized segments have a first circumferentially orientated polarity and each of the plurality of moveable magnetized segments have a second circumferentially orientated polarity.

In third aspect, an actuator for a ventricle assist device (VAD) or a total artificial heart (TAH) includes a driver for generating a first force for driving the VAD or TAH, and a magnetic spring for magnetically applying a second force for driving the VAD or TAH.

In a fourth aspect, an actuator for a ventricle assist device (VAD) or a total artificial heart (TAH) includes a rotatable member, a translatable member for driving the VAD or TAH, a driver for imparting rotary torque to the rotatable member, a magnetic coupling for converting rotary torque of the rotatable member to a first axial force on the translatable member, and a magnetic spring for magnetically applying a second axial force on the translatable member. The magnetic spring comprises a plurality of spaced-apart stationary magnetized segments defining a first plurality of spaced-apart gaps, a plurality of spaced-apart moveable magnetized segments defining a second plurality of spaced-apart gaps, and wherein each of the plurality of moveable magnetized segments is slidable within a respective one of the first plurality of gaps defined by the plurality of stationary magnetized segments.

In a fifth aspect, a ventricle assist device (VAD) includes a housing having a first ventricle, a first diaphragm coupled to the first ventricle for pumping blood therefrom when actuated towards the housing, and an actuator as noted above.

In a sixth aspect, a total artificial heart (TAH) includes a housing having a first ventricle and a second ventricle, a first diaphragm coupled to the first ventricle for pumping blood therefrom when actuated towards the housing, and a second diaphragm coupled to the second ventricle for pumping blood therefrom when actuated towards the housing, and an actuator as noted above.

In an seventh aspect, a method for storing energy includes arranging a plurality of spaced-apart stationary magnetized segments around a circumference to define a first plurality of spaced-apart gaps with each of the plurality of stationary magnetized segments having a first circumferentially orientated polarity, arranging a plurality of spaced-apart moveable magnetized segments around the circumference to define a second plurality of spaced-apart gaps with each of the plurality of moveable magnetized segments having a second circumferentially orientated polarity, and at least one of moving the plurality of moveable magnetized segments between the plurality of stationary magnetized segments and moving the plurality of moveable magnetized segments disposed between the plurality of stationary magnetized segments out of axial alignment with the plurality of stationary magnetized segments.

### BRIEF DESCRIPTION OF THE DRAWINGS

FIG. 1 is a perspective view of one embodiment of a magnetic spring according to the present invention;

FIG. 2 is a cross-sectional view of the magnetic spring of FIG. 1;

FIG. 3 is a cross-sectional view of a second embodiment of a magnetic spring according to the present invention;

FIG. 4 is a partial view of another embodiment of a magnetic spring having a magnetized segment having a tapering cross-section according to the present invention;

FIG. 5 is a partial view of another embodiment of a magnetic spring having a magnetized segment having a tapering cross-section according to the present invention;

FIG. 6 is a perspective view of a plunger incorporating the plurality of moveable magnetized segments of FIG. 1;

FIG. 7 is a perspective view of a housing incorporating the plurality of stationary magnetized segments of FIG. 1;

FIG. 8 is a cross-sectional view of one embodiment of a VAD according to the present invention incorporating the magnetic spring of FIG. 1 wherein solid lines illustrate the pusher plate in a fill position and broken lines illustrate the pusher plate in an eject position; and

FIG. 9 is a graph of the pressure verses time for a circulatory test system and a mathematical simulation;

FIG. 10 is a graph illustrative of an idealized torque verses time for an eject phase and retract phase of an actuator without a magnetic spring;

FIG. 11 is a graph illustrative of an idealized torque verses time for an eject phase and retract phase of an actuator with a magnetic spring; and

FIG. 12 is a schematic cross-sectional view of an embodiment of a total artificial heart incorporating a magnetic spring.

### DETAILED DESCRIPTION OF THE INVENTION

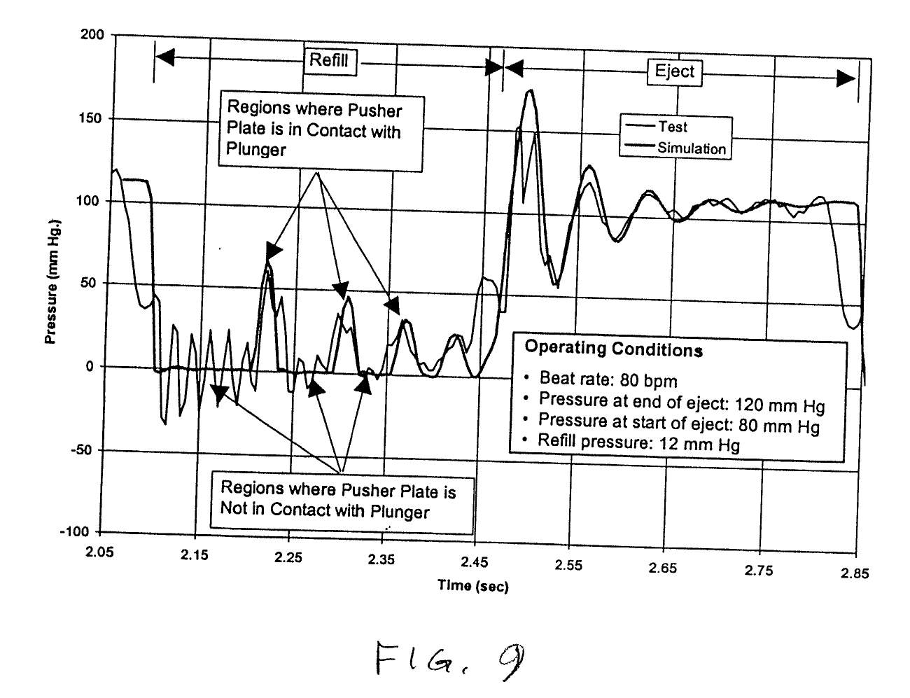

FIGS. 1 and 2 illustrates one embodiment of a magnetic spring 10 in accordance with the present invention. As described in greater detail below, magnetic spring 10 is desirably suitable for use in a pulsatile ventricle assist device (VAD) as shown in FIG. 8 or total artificial heart (TAH) as shown in FIG. 12 in which stored energy in the magnetic spring is used to reduce motor power loses of an actuator during a power stroke of the VAD or TAH.

As best illustrated in FIG. 1, magnetic spring 10 generally includes a first hollow cylinder 20 comprising a plurality of spaced-apart stationary magnetized segments 22 defining a first plurality of spaced-apart gaps 30, and a second hollow cylinder 40 comprising a plurality of spaced-apart magnetized moveable segments 42 defining a second plurality of spaced-apart gaps 50. Each of the plurality of spaced-apart moveable magnetized segments 42 is axially slidable within a respective one of the first plurality of gaps 30 defined between the plurality of spaced-apart stationary magnetized moveable segments 22.

The segments may be formed so that each segment is of equal angular extent. The angular gaps between the segments may be formed so that each gap is equally sized. Desirably, the angular extent of the gaps between segments are slightly larger than that of the magnets so that when the spring is assembled an angular clearance exists between the adjacent magnets from each segment.

In this illustrated embodiment, the plurality of spaced-apart stationary magnetized segments 20 and the plurality of spaced-apart moveable magnetized segments 40 define intermeshed cylinders with a primary spring force being directed along a concentric axis A of the cylinders and the primary spring force being a function of the relative displacement of the moveable magnetized segments relative to the stationary magnetized segments. In this configuration, the magnetic field or flux between the magnetized segments is in a circumferential or tangential direction, e.g. between the magnetized segments.

With reference again to FIG. 2, each of magnetized segments 22 and 42 comprises a circumferentially magnetized arc segment, e.g., the polarity of a magnetized segment being either north and south along an axial longitudinally-extending side portion of the magnetized segment. Placing the same polarity of a moveable magnetized segment and a stationary magnetized segment adjacent each other, as shown in FIG. 2, results in the moveable magnetized segments tending to repel from and move away from the stationary magnetized segments when the stationary and moveable magnetized segments are longitudinally aligned, e.g., an external force is required to force the two sets of magnetized segments together.

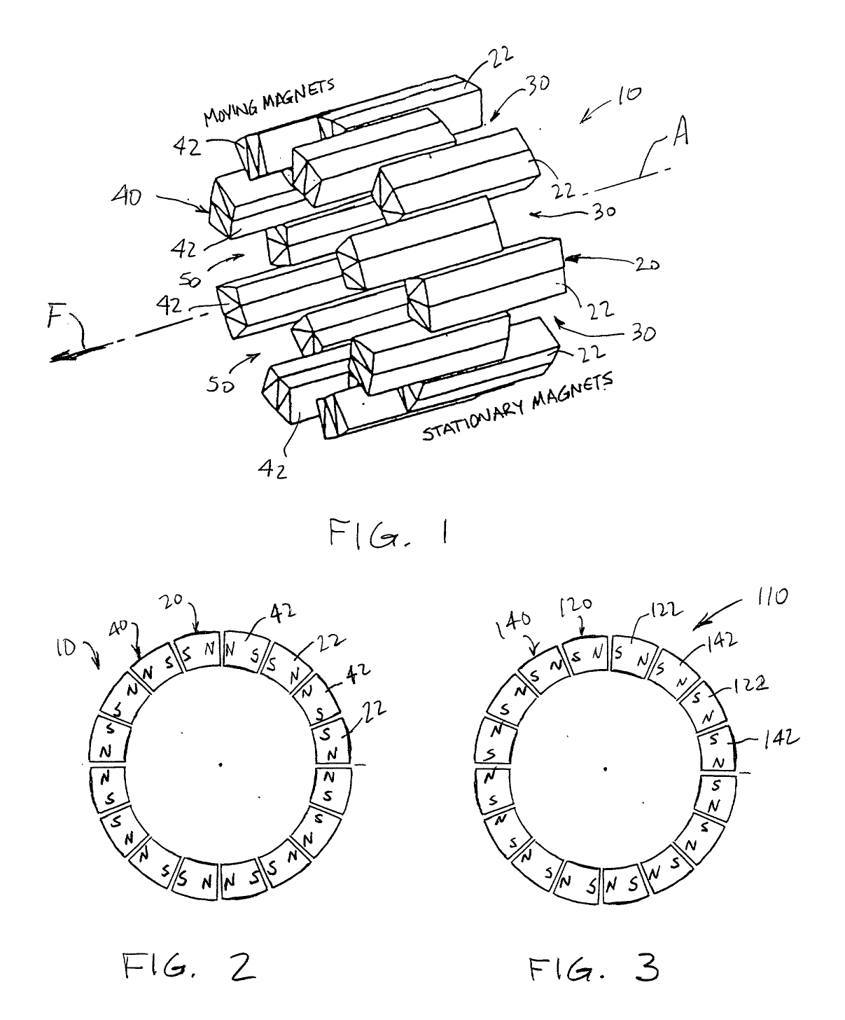

The action of the two magnetized segment sets on one another when aligned results in an axial repulsive force F (or an attractive force for the configuration shown in FIG. 3 and discussed below) between the two magnetized segment sets. Ideally, in the absence of fringing, this repulsion force is given by the equation,

$${f}_{ideal} = \frac{{B}_{r}^{2}}{{μ}_{r}^{2} * {μ}_{o}} * \frac{{p}_{s} {p}_{m}}{{p}_{t}^{2}} * {t}_{m} {p}_{t}$$

where, Bᵣ is the magnet residual induction, μᵣ is the magnet relative recoil permeability, μₒ is the permeability of air, pₛ is the total perimeter of the stationary magnets, pₘ is the total perimeter of the moveable magnetized segments, pₜ is the total available perimeter, and tₘ is the radial thickness of the magnetized segment. If the magnetized segments of the stationary magnetized or in movable magnetized segments have different radial thickness, then tₘ is the radial thickness of the thinner magnetized segments.

With reference to FIG. 3, another embodiment of a magnetic spring 110 generally includes a first cylinder 120 comprising a plurality of spaced-apart stationary magnetized segments 122, and a second cylinder 140 comprising a plurality of spaced-apart moveable magnetized segments 142. In this embodiment, placing opposite polarities of a moveable magnetized segment and a stationary magnetized segment adjacent each other, as shown in FIG. 3, results in the moveable magnetized segments tending to remain longitudinally aligned within the stationary magnetized segments, e.g., an external force is required to force the two sets of magnetized segments away from each other.

Whether the magnetic spring acts in a repulsive force mode (FIGS. 1 and 2) or an attractive force mode (FIG. 3), the force will tend to remain constant over most of the displacement range. The forces will deviate from this constant value as a result of magnetic fringing when the magnet arrays approach full displacement engagement or full disengagement. Different force verses displacement configurations can be obtained by sizing and/or shaping the magnetized segments.

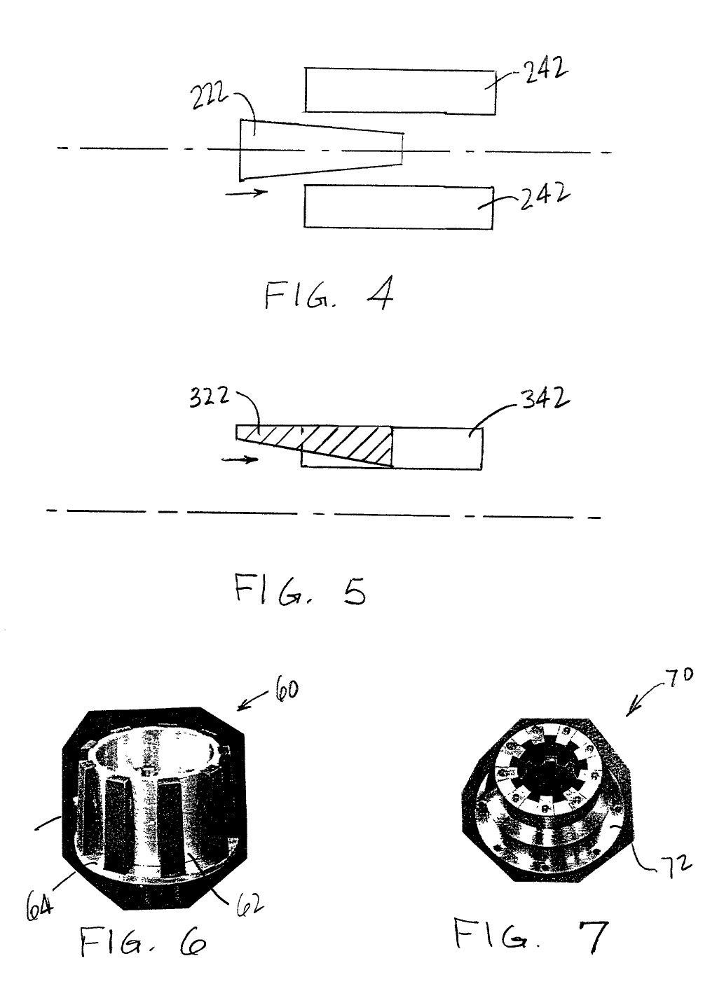

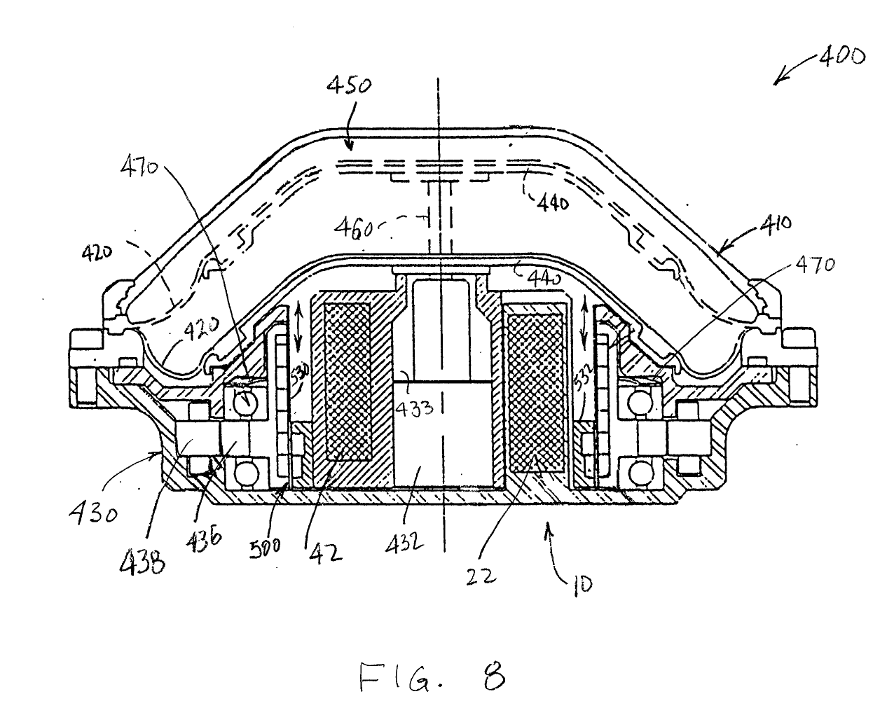

For example, at least one of the plurality of stationary magnetized segments may comprise a longitudinally-extending tapering cross-section, and/or at least one of the plurality of moveable segments may comprises a longitudinally-extending tapering cross-section. The tapering of a magnetized segment may occur, e.g., along the longitudinal length of the segment when viewed normal to the outer tangential surface of the magnetized segment as shown in FIG. 4, and/or along the longitudinal length of the magnetized segments when viewed parallel to the tangential surface of the magnetized segment as shown in FIG. 5.

With reference to FIG. 4 in which similar magnetic poles of the magnetized segments are disposed adjacent to each other, as magnetized segment 222 is moved toward and into the gap between magnetized segments 242, the repelling force is initially low and increases with increased engagement. With reference to FIG. 5 in which similar magnetic poles of the magnetized segments are disposed adjacent to each other, as magnetized segment 322 is moved toward and into the gap between magnetized segments 342, the repelling force is initially large and then decreases with increasing engagement.

From the present description, it will be appreciated by those skilled in the art that other configuration may be employed to vary the force verses displacement relationship of the magnetic spring, e.g., a magnetized segment having a stepped configuration or other configurations. Thus, by varying the shape of the magnetized segments it is possible to tune the magnetic spring, e.g., tailor the force verses the displacement relationship of the magnetized segments.

As shown in FIG. 6, a plunger 60 may be formed with the moveable magnetized segments attached to an outer surface of a hollow cylindrical member 62 having an outwardly-extending flange 64. As shown in FIG. 7, a housing 70 may be formed with the stationary magnetized segments attached to an inner surface a hollow cylindrical member 72. Plunger 60 is receivable within housing 70.

FIG. 8 is a cross-sectional view of one embodiment of a ventricle assist device (VAD) 400 according to the present invention which incorporates magnetic spring 10. As shown in FIG. 8, VAD 400 generally includes a housing 410, a reciprocable diaphragm 420, a pusher plate 440, and an actuator 430 which incorporates magnetic spring 10 and which is operably coupled to pusher plate 440 to drive pusher plate 440 toward the inner surface of housing 410. Reciprocable diaphragm 420 and an inner surface of housing 410 define there between a pumping chamber 450.

With only one pumping chamber, the return stroke of the actuator can be used to store energy in the magnet spring, which reduces the loads and the energy consumption during the VAD power stroke. During the fill cycle, the actuator “cocks” the magnetic spring, and, during ejection, the magnetic spring assists the actuator in emptying the pump. The maximum power requirement of the VAD is thereby reduced compared to a VAD which does not incorporate a magnetic spring. Unlike a coil spring, the force output to the magnetic spring can be tuned to best match the desired ejection load characteristic as discussed above. This, in turn, enables the use of smaller actuator components, while maintaining or increasing life and reliability.

In this illustrated embodiment of actuator 430, actuator 430 includes a linear bearing 432, magnetic spring 10, a rotary-to-axial force energy conversion coupling 500, and a drive motor comprising a motor rotor 436 and a motor stator 438. Stationary linear bearing shaft 433 guides a guide pin 460 afixed to pusher plate 440 toward the inner surface of housing 410 while linear bearing 432 is driven toward the inner surface of housing 410 by energy conversion coupling 500.

Conceptually, the rotary torque-to-axial force (or axial force-to-rotary torque) energy conversion coupling 500 is analogous to a mechanical screw coupling wherein the mechanical thread is replaced by a “magnetic thread” having no contact, wear, or friction between the moving elements of the magnetic coupling. An example of this magnetic thread coupling includes a first magnet member or magnut 530 of the magnet coupling which comprises a cylindrical structure within which a second magnet member or magscrew 532, also a cylindrical structure, resides.

Magnut 530 comprises interleaved magnet sections, or more definitively, a magnetic thread consisting of a spiral wound pair of radially polarized magnets of opposite polarity. Similarly, magscrew 532 comprises a spiral wound pair of radially polarized magnets of opposite polarity. The magnet pairs of the magnut 530 and magscrew 532 tend to align themselves such that magnetic fluxes align with each other. With the two members so aligned, no rotational torque or axial force exists between them. This is the null force position, or the relative position to which the magnet coupling returns when no external forces act on either member.

Where magnut 530 and magscrew 532 are displaced relative to one another in the tangential direction, e.g., by rotating magnut 530, a relative force is generated between the two members, tending to return them to the null position. In this example, this force includes an axial force on magscrew 532 in either of the directions shown by the double headed arrow. This axial force component of the magnet coupling will comprise the force an actuator applies to the blood pump. The tangential component of the axial force generates the torque that the rotary drive motor must overcome to activate the magnetic coupling.

Such a rotary torque-to-axial force energy convertor is disclosed in greater detail in U.S. Pat. No. 5,984,960 issued to Vitale, and U.S. patent application Ser. No. 09/382,143, filed Aug. 24, 1999, entitled “Rotary Torque-To-Axial Force Energy Conversion Apparatus,” which has issued as U.S. Pat. No. ______ to Vitale, in connection with a total artificial heart (TAH). The entire contents of these patents are incorporated herein by reference. The VAD operates similarly, except that only one pump is involved.

The magspring in this design is packaged in the cylindrically shaped space between the inside diameter of the magnut and the outside diameter of the linear bearing. The design allows use of a pure linear bearing and incorporates two Kaydon REALI-SLIM angular contact ball bearings commercially available from Kaydon Corp. of Muskegon, Mich. to radially support the magnut component and absorb the actuator thrust forces. The small cross-section of these bearings allows the drive motor to be fit between the two bearings, while the high thrust capacity of the angular contact construction provides the potential for excellent bearing life and reliability. The use of the magspring reduces the drive torque required from the drive motor, and that, coupled with the increase in motor diameter, permits the reduction in drive motor width required to package it between the two radial bearings.

In operation, when the motor turns, the magscrew moves linearly, as a plunger. Approximately two revolutions of the motor moves the plunger from a refill position to an eject position. The motor then stops, reverses, and moves the plunger back to the refill position. The VAD operates in conjunction with the natural heart and the inlet cannula to the VAD is attached to the left ventricle of the natural heart. During natural heart systole, blood is pumped from the natural heart ventricle into the pump chamber causing the pusher plate to move towards the refill position as pump chamber 450 fills. When the natural heart completes systole and blood flow from the natural heart to the VAD ceases, the control logic senses the end of pusher plate motion and causes the VAD to eject the blood in the pump chamber into the aorta. As noted above, actuator 430 is not directly coupled to pusher plate 440. Thus, after blood from the blood pump is ejected, the filling depends on the natural heart systole. For example, during fill, guide pin 460 is free to slide within the actuator, so diaphragm fill cycle motion is determined by venous pressure, rather than the actuator rate. Control logic senses the velocity or position of the diaphragm, and maintains an actuator speed sufficient to avoid fill cycle contact between pusher plate and actuator, without running so fast that efficiency or operation of the opposite pump is impacted. A TAH, as disclosed below, involves two pumps.

VAD Test and Comparison to Simulation

An experimental VAD was tested in-vitro on a VAD test fixture both with and without a magnetic spring. The magnetic spring was designed to be readily removeable without actuator disassembly. This enabled tests to be performed with identical pump, loop and data acquisition conditions, both with and without the magspring.

The actuator included magnets in the magscrew having an axial width of about 0.125 inch for an equivalent thread pitch of about 0.25 inch (i.e., one revolution of the magnut advances the magscrew 0.25 inch). The mechanical advantage of the magscrew is 989 so that 1 N-m of torque on the magnut results in 989 N of axial force on the magscrew. The pusher plate area in the blood pump was 45 cm², and, consequently, a pump chamber pressure of 100 mm Hg translates to a pusher plate force of 60 N and a magnut torque of 60/989 or 0.0607 N-m. The magnetic interaction between the two elements allows force generation to occur across a 0.65 mm (0.016 inch) air gap between the smooth adjacent surfaces of the magscrew and magnut.

The drive motor used was an Inland motor, model number RBE-018100A00 commercially available from Kollmorgan Inc., Inland Motor Division of Radford, Va. having a motor resistance of 1.22 ohm, a motor inductance of 760 mH, a peak motor constant of 0.0856 N-m/A, and an average motor constant of 0.0827 N-m/A between motor commutations. For a steady-state torque of 0.0607 N-m, the motor requires an average current of 0.734 A.

Design analysis of a magnetic spring of appropriate size and force characteristics for a VAD application was performed using a time-stepping dynamic simulation code. To simplify matters, a straight-line force-to-displacement characteristic was assumed. Therefore, the spring force was defined by mean force and force slope. The force slope is the force at full eject minus the force at full refill, divided by the corresponding magspring displacement. A negative value of slope means that the spring force is higher when the spring is retracted to the refill position, as compared to its force at the eject position. Based on the results of a sizing analysis, for a 120/80 mm Hg blood pressure variation, a magspring with a mean force of 30 N and a positive force slope of 2592 N/m was selected. The magnetic segments were neodymium boron iron magnets with an energy product of 39 M gauss oersted. The housing was formed from aluminum.

A VAD bench-test loop was used during the test. Water was used for the pumping fluid. The inlet of the VAD was plumbed to the outlet of a supply tank. The outlet of the VAD was connected to the aortic compliance chamber where back pressures could be adjusted. The outlet of the compliance chamber was connected to the tank inlet. A flow meter and variable restrictor were placed in this line. A computer-based data acquisition system was used to acquire data from the following: a pressure sensor attached to the pump housing; a Linear Velocity Displacement Transducer (LVDT) attached to the magspring plunger; and a current transformer on one motor coil. Supply voltage to the motor was also recorded.

The VAD motor was controlled manually, with beat rate set by a square wave generated by an external oscillator. The only operating mode possible with the controller setup was a fixed-rate cycling. The simulation validation consisted of two corresponding parts: 1) a comparison of the pumping segment (specifically, the pressure variations within the pump chamber) with test data, and 2) a comparison of the actuator (specifically the predicted motor current, voltage, power input, and power loss) with test data. The validations, which are described below, were conducted at the following operating conditions; 80 bpm beat rate, 120 mm Hg pressure at end of eject, 80 mm Hg pressure at start of eject, 12 mm Hg refill pressure.

The simulation included the pump chamber, the inlet and outlet valves, and the inlet and outlet fluid cannula. An important aspect of the validation process was the evaluation of the model parameters required to predict the performance of an optimized VAD. The measurement of pump chamber pressure was made using a Validyne Model 15-DP56 pressure transducer available from Validyne Engineering Corp. of Northridge, Calif. with a 3-dB response frequency of 1000 Hz.

FIG. 9 compares the pressure verses time for a circulatory test system and a mathematical simulation over one pump beat. The beat depicted in FIG. 9 can be conveniently divided into two regions: 1) refill, during which fluid flows from the inlet chamber of the VAD test loop into the pump chamber in which the chamber pressure in this region is low (0 to 70 mm Hg), and 2) eject, during which fluid is moved from the pump chamber into the aortic compliance chamber and exit flow restriction in which the chamber pressure in this region is high (50 to 174 mm Hg).

During eject, the aortic compliance chamber was set to cause the discharge pressure to vary from 80 mm Hg at the beginning of eject to 120 mm Hg at the end of eject. The variable exit flow restriction is downstream of the compliance chamber and set to achieve the mean discharge pressure of 100 mm Hg. As shown in FIG. 9, the mathematically calculated initial pressure rise (175 mm Hg) overshoots the initial cannula pressure (80 mm Hg) by 95 mm Hg. This overshoot is required to overcome the inertia of the fluid in the exit cannula as cannula fluid motion is initiated. The compliance of the pump reduces the amplitude of this inertial pressure spike and results in the subsequent oscillation of chamber pressure as the chamber compliance and fluid inertia interact. The chamber pressures oscillation subsequent to the initial pressure overshoot are characterized by the oscillation frequency and rate of decay.

During the refill portion of the pump cycle, the pressure at the entrance to the inlet cannula is constant at 12 mm Hg. Blood inertia causes the pusher plate to separate from the plunger at the end of the eject stroke, when the plunger reverses direction. While the pusher plate is separated from the plunger, the refill pressure acts to reverse its direction and blood begins to refill the pump chamber. At 12 mm Hg, the refill pressure causes the pump chamber to refill faster than the plunger is retracting, and, consequently, the pusher plate re-contacts the plunger at T=2.2 sec. The inertia of the blood in the inlet cannula now causes the pump chamber diaphragm to stretch so that the displacement of the blood in the inlet cannula now exceeds the displacement of the pusher plate. The chamber pressure rise associated with the diaphragm stretch forces the pusher plate off the plunger a second time at T=2.22 sec, and the refill pressure causes the pusher plate to re-contact the plunger a second time at T=2.28 sec.

The circulatory test system did not include sensors capable of measuring the small relative motions of the pusher plate and the plunger. However, the effects of the displacement behavior can be seen indirectly in terms of its effect on chamber pressure. That is, because the pusher plate is so light when it separates from the plunger, the pressure in the pump chamber momentarily drops to zero. This tendency is clearly seen in the chamber pressure history presented in FIG. 9. As this figure shows, a close correlation between the mathematical simulation and circulatory test system data is observed.

A portion of the mathematical simulation involved predictions of motor current, voltage, power input, and power loss. However, attempts to directly measure motor voltage were foiled due to the high-frequency pulse width modulation (PWM) operation of the Inland motor controller. The potted, encapsulated construction of the controller also precluded measuring voltage just prior to the PWM. The only location to conveniently measure voltage was at the input connector to the controller, a location that also included all other controller electronics. The power requirements of these electronics obscured the voltage parameter measurement. As a result, it was not possible to measure motor input voltage to calculate motor input power. A data acquisition system was utilized to directly measure current flow dissipated by the motor coils and to calculate the associated power loss. Motor power loss is determined using current flow of the motor coils and calculating the associated power loss by numerically integrating the I²R losses over each commutation.

The actual verses simulated motor coil power loss of the actuator as follows:

| | Motor Coil Power Loss | Motor Coil Power Loss |
|---|---|---|
| | Without Magspring | With Magspring |
| Test | 1.77 | 1.55 |
| Analysis | 1.78 | 1.54 |

The correlation is excellent and bodes well for an accurate prediction of the performance of a VAD motor and actuator that is sized to make optimum use of a magspring.

Desirably, the VAD is packaged as a 108-mm diameter, a 58-mm thickness, and a 950-g weight which provides 4.0 Ipm at 80 bpm and require only 1.35 W of input electrical power to pump this flow against a 120/80 mm Hg aortic blood pressure. Such a VAD results in a very low power, low wear and rugged magscrew/magspring actuator.

Proposed VAD Design

A magnetic rotary Torque-To-Axial force energy conversion coupling with a magnetic spring is desirably suitable for use as an actuator in Cleveland Clinic Foundation (CCF) implanted blood pumps like the biolized blood pump and actuator percutaneous vent line (to vent the gas space behind the pusher plate) and the percutaneous power and sensor leads which connect the implanted VAD to the external controller.

In the CCF blood pump, the blood pump housing is made from a biocompatible carbon fiber epoxy composite material. The diaphragms are compression-molded from HEXSYN rubber, a high-flex-life polymer developed by the Goodyear Tire & Rubber Company. The blood contacting surfaces of both the diaphragm and pump housing are textured and then coated with a biolized layer consisting of glutraraldehyde cross-linked, collagen-based gelatin. Inflow and outflow valves are tri-leaflet and fabricated from bovine pericardium. The use of natural tissue valves and the biolized layer eliminate the need for anticoagulation. The pump vent and cables are of conventional dacron-covered design.

The drive motor, as discussed above, is a brushless DC motor. Desirably the drive motor has 24 poles, a 64-mm rotor magnet diameter, a 5-mm rotor magnet width, and a 78-slot stator. To conserve space and minimize motor width, the stator coil end turns can be wound over the top of the stator, rather than tangentially along the side. The stator laminations and rotor solid back iron are desirably laser-cut and the inter-lamination insulation can be provided by oxidizing the surfaces during the heat-treating process reduced to minimize motor size.

Hall-effect sensors commercially available from Allegro MicroSystems Inc. of Worcester, Mass. are desirably used to measure the pump pusher plate displacement. These sensors, mounted internal to the VAD, can view a magnet mounted on the back side of the pusher plate. They can be mounted on their own local circuit board, which will also contain a voltage amplifier to boost the probe output to a voltage level consistent with the associated data acquisition devise. Once installed, the sensors are calibrated in place by connecting a micrometer head to the pusher plate and monitoring the sensor voltage output as a function of displacement. This data will be used to construct a linearization curve, which will then be used in the data acquisition software to convert the sensor readings to actual displacement. Since space requirements are not as stringent for the VAD actuator compared to a TAH, desirably a Hall-effect sensor commutation is used which is more efficient than sensorless commutation. Such commutation can be based on a type MC33035 motor controller chip, which commutates the motor based on the input from three Hall-effect sensor attached to the motor stator. The chip provides the drive inputs to a three-phase bridge assembled from six MOSFET transistors.

The radial and linear bearings may be two RBC KA020AR0 angular contact ball bearings and one LSAGT-6 angular, linear ball spline bearing, commercially available from Nippon Thompson Co. Ltd. of Tokyo, Japan. The radial bearings support the magnut assembly and allow rotation while preventing axial motion. The linear bearing enables reciprocation of the magscrew and the magnetic spring while preventing their rotation. The primary load on the radial bearings is the thrust associated with pusher plate eject and refill. Without the magnetic spring, the thrust load is high during eject and low during refill. In this case, the thrust on the radial bearings can be kept unidirectional by slightly offsetting the motor rotor in the direction of the pump chamber so as to generate a small and constant magnetically induced axial force on the radial bearings in the same direction as the normal eject force. This small force prevents thrust reversals on the bearing during refill, and precludes the need for bearing pre-load springs. With the magnetic spring in place, however, the thrust load situation changes. The bearing thrust load is now moderate during eject, when force from the magnetic spring reduces the force, and during refill, when the magnetic spring force acts alone. This thrust load acts in one direction during eject and in the opposite direction during refill. Because of the thrust force present during pump refill, the motor offset approach is no longer viable and a pre-load washer-type spring 470 (FIG. 8) can be used instead.

The thrust load generated by the pre-load spring causes the thrust load with the magnetic spring to be similar to thrust load without the magnetic spring (i.e., higher during eject and lower during refill). Radial bearing load, estimated assuming constant 80-bpm operation into a blood pressure of 160/120 mm Hg, is equivalent to 84 N during eject and near zero during refill.

With the magnetic spring in place, the linear bearing will experience moderate torques during both eject and refill. For ejection into a blood pressure of 160/120 mm Hg, the average linear bearing torque will be +0.066 N-m during eject and −0.033 N-m during eject.

A control approach for the VAD is based on the velocity. This involves determining the derivative of the fill stroke, and triggering eject when the filling velocity slows to near zero. This approach maintains a good counter-pulsation operation with a natural heart.

Advantageously, a controller is capable of easy reconfiguration in order to apply the alternative control strategies described above. For example, the controller desirably provides two basic functions. First, is directed to the motor power and control. As described earlier, the controller utilizes Hall probe commutation and motor controller chip MC33035 to run the brushless DC motor. The controller receives signals from three Hall-effect sensors attached to the motor stator. These signals can be conditioned and used as input to the MC33035. The chip provides the drive inputs to a three-phase bridge assembled from six MOSFET transistors and associated free-wheeling diodes. This bridge output can be connected to the three phases of the drive motor. The second is directed to the actuator control. One or two Hall-effect sensors can be positioned to sense end-of-stroke from a magnet mounted on the pusher plate. These signals will be conditioned by the controller software and input to the MC33035 to start and stop the motor, apply the motor brake function, and reverse the direction of the motor. The circuitry for these functions are desirably contained on a printed circuit board. This circuitry, along with motor and electronics power supplies, can be housed in a splash-proof box having start/stop controls, switches for manual and automatic operation, and a read-out of bpm. BNC electrical connectors can also be provided for monitoring motor voltage and current, as well as diaphragm motion.

The above control approaches can be suitably implemented on a PC using a LabView software package by National Instruments with associated input and output boards. The amplified voltage output of the pusher plate Hall probe can be input to the LabView data acquisition system and converted by LabView software into actual pusher plate displacement.

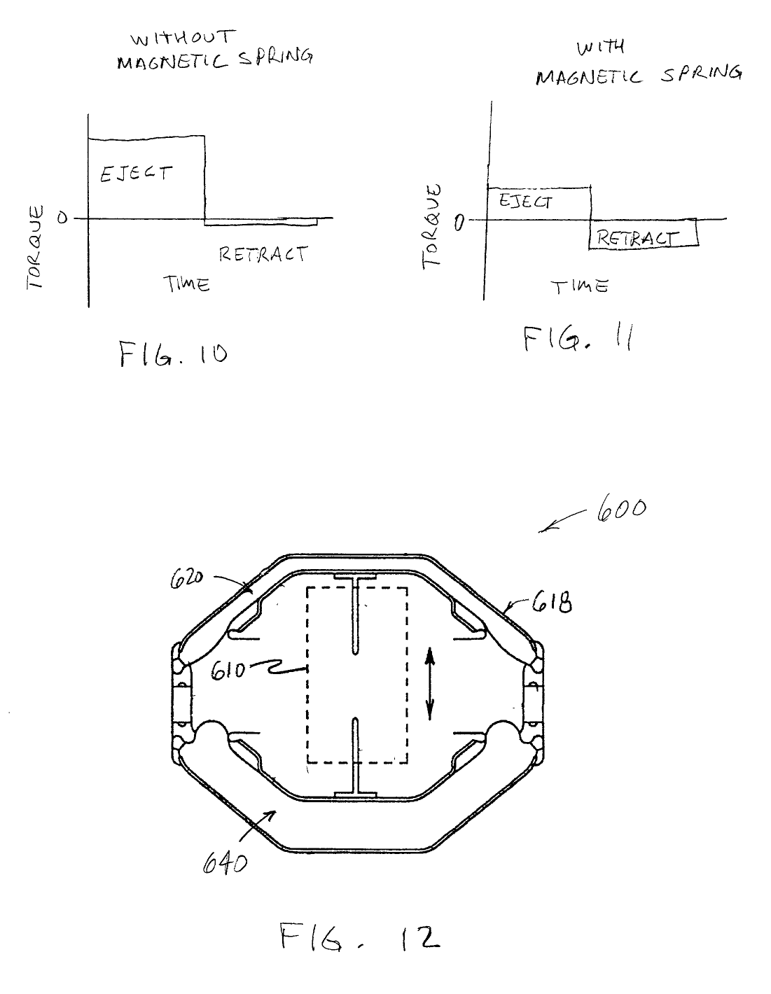

With reference to FIG. 10, a total artificial heart (TAH) 600 having an actuator 610 which incorporates a magnetic spring 10 of FIG. 1 is illustrated. As shown in FIG. 10, TAH 600 includes a left or ventricle blood pump 120 and a right blood pump or ventricle 640 within a housing 618. The magnetic spring can be designed to exploit the asymmetric pumping forces required of the TAH.

To restate, the present invention broadly comprises in one embodiment a magnetic spring for use in connection with a rotary torque-to-axial force (or axial force-to-rotary torque) energy conversion apparatus, with one significant application thereof comprising an actuator for a ventricle assist device (VAD) or for a total artificial heart (TAH). An actuator in accordance with this invention employs a magnetic coupling and magnetic spring which totally eliminate contact, wear and friction between the principal moving elements of the actuator. The magnetic coupling, which consists of a helically wound pair of radially polarized magnets of opposite polarity, takes place through a thin isolation wall, permitting important bearing components and their lubricants to be sealed. These components, along with the drive motor, are therefore also isolated from the humid inter-pump space. The new actuator is expected to provide much longer life, lower heat generation, and increased reliability, compared to existing systems.

Thus, while various embodiments of the present invention have been illustrated and described, it will be appreciated to those skilled in the art that many changes and modifications may be made thereunto without departing from the spirit and scope of the invention.

1. A magnetic spring comprising:
a plurality of spaced-apart stationary magnetized segments defining a first plurality of spaced-apart gaps;
a plurality of spaced-apart moveable magnetized segments defining a second plurality of spaced-apart gaps; and
wherein each of said plurality of moveable magnetized segments is slidable within a respective one of said first plurality of gaps defined by said plurality of stationary magnetized segments.

2. The magnetic spring of claim 1 wherein said plurality of stationary magnetized segments and said plurality of moveable magnetized segments are disposed along an arc.

3. The magnetic spring of claim 1 wherein said plurality of stationary magnetized segments and said plurality of plurality of moveable magnetized segments generally define a hollow cylinder.

4. The magnetic spring of claim 1 wherein at least one of said plurality of stationary magnetized segments comprises a tapering cross-section.

5. The magnetic spring of claim 1 wherein at least one of said plurality of moveable magnetized segments comprises a tapering cross-section.

6. The magnetic spring of claim 1 wherein longitudinally-extending side portions of said plurality of stationary magnetized segments comprise a first orientated polarity and longitudinally-extending side portions of said plurality of moveable magnetized segments comprise a second orientated polarity.

7. The magnetic spring of claim 1 wherein said plurality of stationary magnetized segments and said plurality of moveable magnetized segments when aligned within said gaps tend to move away from each other.

8. The magnetic spring of claim 1 wherein said plurality of stationary magnetized segments and said plurality of moveable magnetized segments when aligned within said gaps tend to remain aligned.

9. A magnetic spring comprising:
a plurality of spaced-apart stationary magnetized segments disposed along an arc about an axis and defining a first plurality of spaced-apart gaps;
a plurality of spaced-apart moveable magnetized segments disposed along said arc and defining a second plurality of spaced-apart gaps;
wherein each of said plurality of moveable magnetized segments is axially slidable within a respective one of said first plurality of gaps defined by said plurality of stationary magnetized segments; and
wherein each of the plurality of stationary magnetized segments have a first circumferentially orientated polarity and each of the plurality of moveable magnetized segments have a second circumferentially orientated polarity.

10. The magnetic spring of claim 9 wherein said plurality of stationary magnetized segments and said plurality of moveable magnetized segments generally define a hollow cylinder.

11. The magnetic spring of claim 9 wherein at least one of said plurality of stationary magnetized segments and said plurality of moveable magnetized segments comprises an axially tapering cross-section when viewed normal to an outer tangential surface of the at least one magnetized segment.

12. The magnetic spring of claim 11 wherein at least one of said plurality of stationary magnetized segments and said plurality of moveable magnetized segments comprises an axially tapering cross-section when viewed parallel to an outer tangential surface of the at least one segment.

13. The magnetic spring of claim 9 wherein said first circumferentially orientated polarity is the same as the second circumferentially orientated polarity so that said plurality of stationary magnetized segments and said plurality of moveable magnetized segments when aligned within said gaps tend to move away from each other.

14. The magnetic spring of claim 9 wherein said first circumferentially orientated polarity is opposite of the second circumferentially orientated polarity so that said plurality of stationary magnetized segments and said plurality of moveable magnetized segments when aligned within said gaps tend to remain aligned.

15. An actuator for a ventricle assist device (VAD) or a total artificial heart (TAH), said actuator comprising:
a driver for generating a first force for driving the VAD or TAH; and
a magnetic spring for magnetically applying a second force for driving the VAD or TAH.

16. The actuator of claim 15 wherein said magnetic spring comprises a plurality of spaced-apart stationary magnetized segments defining a first plurality of spaced-apart gaps, a plurality of spaced-apart moveable magnetized segments defining a second plurality of spaced-apart gaps, and wherein each of said plurality of moveable magnetized segments is slidable within a respective one of said first plurality of gaps defined by said plurality of stationary magnetized segments.

17. The actuator of claim 16 wherein said plurality of stationary magnetized segments and said plurality of plurality of moveable magnetized segments generally define a hollow cylinder.

18. The actuator of claim 16 wherein at least one of said plurality of stationary magnetized segments and said plurality of moveable magnetized segments comprises a tapering cross-section.

19. An actuator for a ventricle assist device (VAD) or a total artificial heart (TAH), said actuator comprising:
a rotatable member;
a translatable member for driving the VAD or TAH;
a driver for imparting rotary torque to the rotatable member;
a magnetic coupling for converting rotary torque of the rotatable member to a first axial force on the translatable member; and
a magnetic spring for magnetically applying a second axial force on the translatable member, said magnetic spring comprising a plurality of spaced-apart stationary magnetized segments defining a first plurality of spaced-apart gaps, a plurality of spaced-apart moveable magnetized segments defining a second plurality of spaced-apart gaps, and wherein each of said plurality of moveable magnetized segments is slidable within a respective one of said first plurality of gaps defined by said plurality of stationary magnetized segments.

20. The actuator of claim 19 wherein said plurality of stationary magnetized segments and said plurality of moveable magnetized segments generally define a hollow cylinder.

21. The actuator of claim 19 wherein at least one of said plurality of stationary magnetized segments and said plurality of moveable magnetized segments comprises a tapering cross-section.

22. The actuator of claim 19, wherein said magnetic coupling comprises a first permanent magnet comprising part of said rotatable member and a second permanent magnet comprising part of said translatable member, and wherein said first permanent magnet comprises interleaved, helical magnet sections of alternating polarities, and wherein said second permanent magnet comprises interleaved, helical magnet sections of alternating polarities.

23. The actuator of claim 22, wherein said actuator is designed to reside within a Cleveland Clinic—type total artificial heart having a first diaphragm at a first ventricle and a second diaphragm at a second ventricle, and wherein said driver comprises a permanent magnet rotary motor which imparts oscillating motion to the rotatable member producing an oscillating rotary torque at the first permanent magnet that in turn produces reciprocating axial movement in the second permanent magnet, and hence the translatable member, said reciprocating axial movement being employed to alternately actuate the first diaphragm of the first ventricle and the second diaphragm of the second ventricle.

24. A ventricle assist device (VAD) comprising:
a housing having a first ventricle;
a first diaphragm coupled to the first ventricle for pumping blood therefrom when actuated towards said housing; and
an actuator of claim 15 for actuating said first diaphragm.

25. A ventricle assist device (VAD) comprising:
a housing having a first ventricle;
a first diaphragm coupled to the first ventricle for pumping blood therefrom when actuated towards said housing; and
an actuator of claim 19 for actuating said first diaphragm.

26. A total artificial heart (TAH) comprising:
a housing having a first ventricle and a second ventricle;
a first diaphragm coupled to the first ventricle for pumping blood therefrom when actuated towards said housing, and a second diaphragm coupled to the second ventricle for pumping blood therefrom when actuated towards said housing; and
an actuator of claim 15 for actuating said first diaphragm and said second diaphragm.

27. A total artificial heart (TAH) comprising:
a housing having a first ventricle and a second ventricle;
a first diaphragm coupled to the first ventricle for pumping blood therefrom when actuated towards said housing, and a second diaphragm coupled to the second ventricle for pumping blood therefrom when actuated towards said housing; and
an actuator of claim 19 for actuating said first diaphragm and said second diaphragm.

28. A method for storing energy, the method comprising:
arranging a plurality of spaced-apart stationary magnetized segments around a circumference to define a first plurality of spaced-apart gaps, each of the plurality of stationary magnetized segments having a first circumferentially orientated polarity;
arranging a plurality of spaced-apart moveable magnetized segments around the circumference to define a second plurality of spaced-apart gaps, each of the plurality of moveable magnetized segments having a second circumferentially orientated polarity; and
at least one of moving the plurality of moveable magnetized segments between the plurality of stationary magnetized segments and moving the plurality of moveable magnetized segments disposed between the plurality of stationary magnetized segments out of axial alignment with the plurality of stationary magnetized segments.

29. The method of claim 28 wherein the first circumferentially orientated polarity is the same as the second circumferentially orientated polarity so that the plurality of stationary magnetized segments and the plurality of moveable magnetized segments when aligned within the gaps tend to move away from each other.

30. The method of claim 28 wherein the first circumferentially orientated polarity is the opposite of the second circumferentially orientated polarity so that said plurality of stationary magnetized segments and said plurality of moveable magnetized segments when aligned within said gaps tend to remain aligned.

31. The method of claim 28 wherein at least one of the plurality of stationary magnetized segments and plurality of moveable magnetized segments comprises a tapering cross-section.
